(19) United States
(12) Patent Application Publication
(10) Pub. No.: US 2022/0054384 A1
(43) Pub. Date: Feb. 24, 2022

(54) MICROBIOSTATIC SOAP THAT REDUCES THE TRANSMISSION OF MICROBES

(71) Applicant: GPCP IP Holdings LLC, Atlanta, GA (US)

(72) Inventors: Brian Patrick Argo, Greenville, WI (US); Reynee Wynne Kachur, Neenah, WI (US); Yin Zheng Hessefort, Menasha, WI (US)

(73) Assignee: GPCP IP Holdings LLC, Atlanta, GA (US)

(21) Appl. No.: 17/312,551

(22) PCT Filed: Nov. 6, 2019
(86) PCT No.: PCT/US2019/060026

§ 371 Date: Jun. 10, 2021

Related U.S. Application Data
(60) Provisional application No. 62/756,791, filed on Nov. 7, 2018.
(60) Provisional application No. 62/806,854, filed on Feb. 17, 2019.

(51) Int. Cl. A61K 8/44; A61Q 17/00; A61Q 19/10; A61K 8/46; A01N 47/12; A01N 41/08

(52) U.S. Cl. CPC A61K 8/442; A61Q 17/005; A61Q 19/10; A61K 2800/5426; A01N 47/12; A01N 41/08; A61K 2800/524; A61K 8/463

(57) ABSTRACT

The present disclosure is directed to a microbiostatic cleansing composition for use on skin and hard surfaces that reduces microbe resistance. The cleansing composition may be, for example, bacteriostatic or virostatic and includes a release agent that entangles the microbes without killing them allowing them to be removed from skin and hard surface in an inactive state.

This disclosure relates to a microbiostatic composition for cleaning and decontaminating skin and hard surfaces. The disclosure also relates to a hand and surface cleaning composition that is effective at removing a wide variety of the microbes that it encounters and will not contribute to the antibiotic resistance of those microbes. The disclosure further relates to a cleaning corn position that puts microbes into stasis, allowing them to be neutralized and removed. The disclosure still further relates to a method of preventing microbial resistance by using a release agent to disrupt microbe binding making it possible to remove microbes from skin and surfaces without killing them.

Medical professionals have long touted that frequent hand washing reduces the spread of viruses and bacteria. Despite increased vigilance, on the order of 80% of infections are still spread by hand to hand contact, making safe effective hand cleaners paramount in the fight to reduce the spread of disease. Current market products, while prolific, possess substantial limits on their efficacy.

Sanitizing compositions and anti-microbial soaps have become ever present in most public establishments in recent years. Hospitals, schools, office buildings, and private homes are all using anti-microbial rinse-off or rinse-free products to keep everyone safer from bacterial and viral infections. Sanitizing compositions, for example, rinse-free gel hand sanitizers, generally use high alcohol contents to attain their anti-microbial activity and kill microbes. Rinse-free compositions are not formulated to remove soil and therefore, many establishments use both rinse-free formulations, as well as rinse-off formulations to address both cleaning and sanitizing. Rinse-off cleansers use a variety of anti-microbial agents that, when combined with the mechanical action of washing, significantly reduce bacteria and viruses found on the skin.

Current commercial rinse-off sanitizers can include one or more anti-microbials, anti-bacterials, germicides, etc. and generally use active ingredients chosen from one or more of iodine compounds, peroxide and per-oxygen compositions, alcohols, phenolics, quaternary ammonium compounds, or chlorine compounds. The aim or each of these ingredients is to kill the microbe.

Additionally, all of these anti-microbials when used in sanitizing products cause skin irritation problems. The high levels of anti-microbial actives needed to attain commercially suitable activity are a primary cause of the irritation experienced upon frequent use. Generally, when anti-microbial products irritate the skin, the user applies the product less frequently. Failure to apply the product as often as needed, increases the likelihood of microbial contamination and the likelihood of developing increased microbial resistance. Ideally, a cleansing product or decontaminating product would be efficacious in a short period of time so that microbes are rendered harmless in the time most people take to wash their hands. In addition, an ideal composition should remain gentle and non-drying to the skin, with little or no irritation.

Studies on the efficacy of sanitizing compositions against specific viruses and bacteria abound. No single composition shows activity as against all of the most common bacteria and viruses. Recently, one of the most prevalent anti-microbials, Triclosan®, has found disfavor and is being removed from consumer products based on a lack of proven efficacy above that of basic soap and water. So, there continues to be a search for sanitizing compositions that are non-hazardous, environmentally friendly, highly effective, and non-irritating.

In addition to these requirements, a need exists for an antimicrobial that has broad application and doesn't contribute to microbial resistance. The prolific use of anti-microbials and antibiotics has caused microbes to adapt and become resistant to the chemicals we use to kill them. Alternative methods and materials to dean and sanitize without making the microbes further resistant have been highly sought after by the Food and Drug Administration. The cleaning composition as described herein is microbiostatic and in at least one embodiment, forms a barrier layer to provide continued efficacy against microbes entering or exiting the skin of the user. As used herein “microbiostatic” refers to the removal of a microbe by one or more means that cause the microbe to be in stasis, i.e., unable to infect, but without kill ng the microbe.

To understand the role that resistance plays, consider a simple example using bacteria. Bacteria found on the skin can be divided into two groups: resident and transient bacteria. Resident bacteria are Gram positive bacteria which are established as permanent microcolonies on the surface and outermost layers of the skin and play an important, helpful role in preventing the colonization of other, more harmful bacteria and fungi. Transient bacteria are bacteria which are not part of the normal resident flora of the skin, but can be deposited when airborne contaminated material lands on the skin or when contaminated material is brought into physical contact with it. Transient bacteria are also typically divided into Gram positive and Gram negative subclasses. Gram positive bacteria include pathogens such as Staphylococcus aureus, Streptococcus pyogenes, Clostridium difficile, enterococci, including vancomycin-resistant enterococci, (VRE), and Clostridium botulinum. Gram negative bacteria include pathogens such as Salmonella, Escherichia coli, Klebsiella, Haemophilus, Pseudomonas aeruginosa, Proteus and Shigella dysenteriae. Gram negative bacteria are generally distinguished from Gram positive by an additional protective cell membrane which generally results in the Gram negative bacteria being less susceptible to topical antibacterial actives.

Bacteria can become resistant by a number of different methods. They can be naturally resistant, they can mutate, or they can acquire the resistance from another bacteria. Regardless of how they become resistant, once resistant bacteria are part of a colony, either a different antibiotic or a stronger antibiotic must be used to successfully kill the resistant bacteria. If the antibiotic is not strong enough to kill the colony, but is strong enough to kill the non-resistant bacteria, then the bacterial colony is left with a majority of resistant bacteria, which then proliferate. Because the instant compositions do not kill the bacteria, the bacterial colony should remain unchanged giving rise to the development of less resistant bacteria.

Clostridium difficile, is one example of a resistant bacteria that proliferates primarily in healthcare environments. C. difficile is a rod-shaped bacteria that is difficult to kill as it forms heat resistant spores that can live for long periods on surfaces. Most C. difficile infections are transmitted by healthcare workers touching contaminated surfaces and transferring the spores to other surfaces. These acid-resistant spores are ingested and pass to the human colon unharmed. As strains of C. difficile become more resistant to known antibiotics, infections become harder and harder to treat. Once C. difficile infects someone, the bacteria colonizes in the patient's colon and may cause symptoms or be non-symptomatic. However, when that patient takes antibiotics for any reason, the antibiotics naturally kill some of the flora in the patient's gut causing the more resilient C. difficile in the colon to flourish taking over the nutrients and space occupied by the now dead flora. As C. difficile flourishes, it becomes symptomatic causing diarrhea making it more easily transmitted.

Alcohol based hand sanitizers do not kill C. difficile spores and early data suggests that, removal or inactivation of C. difficile spores using soap and water is more challenging than removal or inactivation of other common pathogens. Commonly, surfaces need to be cleaned with hypochlorite or bleach solutions to effectively prevent transmission. The cleaning composition as described herein is particularly useful for removing resistant pathogen, like C. difficle. Since the cleaning solution inactivates and removes the pathogen, unlike current hand sanitizers or anti-microbial soap, it remains effective against resilient pathogens. In addition, since the cleaning solution effectively entangles the pathogen and repels it from the surface, both surface cleaning and hand cleaning are more effective.

While often discussed herein in terms of bacteria and bacterial resistance, the composition as described can be applied to reduce antifungal resistance, antiviral resistance, and the like, depending upon the microbe being treated. Since the compositions as described herein do not affect either the membrane or the proteins extruded from the microbe, the microbes enter stasis, but are otherwise intact. The composition as described is mild, effective, fast-acting, doesn't contribute to drug resistance, and provides a protective skin coating, making it ideal for widespread use in soaps and sanitizers.

### SUMMARY OF THE INVENTION

The disclosure is directed to microbiostatic compositions and their use as soaps and cleaners to remove dirt and microbes without contributing to the development of microbial resistance.

According to one embodiment, the disclosure relates to a microbiostatic cleansing composition comprises at least one release agent that entangles one or more microbes upon contact putting them in stasis.

According to another embodiment, the disclosure relates to a microbiostatic cleansing composition consisting essentially of at least one release agent that entangles one or more microbes upon contact putting them in stasis.

According to yet another embodiment, the disclosure relates to a microbiostatic cleansing composition comprising a release agent chosen from one or more zwitterionic surfactants or cationic polymer surfactants.

According to still another embodiment, the disclosure relates to a method for cleansing and decontaminating a surface comprising applying a microbiostatic cleansing composition to the skin, wherein the cleansing composition does not kill the microbes present on the skin; and rinsing the microbes off the skin. The method further includes depositing a substantive layer on the skin or surface to provide extended protection to the surface.

A better understanding of the various disclosed system and method embodiments can be obtained from the following detailed description.

### BRIEF DESCRIPTION OF THE DRAWINGS

FIG. 1 illustrates a soiled surface coming into contact with a cleaning composition as described.

FIG. 2 illustrates how the cleaning composition as described coats the surface with a substantive layer while entangling the dirt and microbes.

FIG. 3 illustrates how the cleaning composition as described entangles the dirt and microbes to create positively charged structures that are repelled by the positively charged substantive layer that coats the surface.

FIG. 4 illustrates how the entangled microbes are separated from the release agent as they are rinsed into a waste water environment.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. It is to be fully recognized that the different teachings of the embodiments discussed below may be employed separately or in any suitable combination to produce desired results. Finally, one skilled in the art will understand that the following description has broad application, and the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not structure or function.

As used in the following discussion and in the claims, the terms “including” “is”, “comprising”, “containing”, etc. are used in an open-ended fashion, and thus, should be interpreted to mean “including, but not limited to.” If closed language is included, “consisting,” and “consisting essentially of” it will be given its art recognized meaning. As used herein, “consisting essentially of” means the composition contains no ingredient in such a proportion as to prevent stasis of, disrupt stasis of, or kill a microbe.

As used herein “stasis” means a state of suspended growth and activities, e.g., unable to infect, but revivable, for example, in the case of bacteria, or reanimatable, for example, in the case of a virus.

“Decontaminate,” as used herein, is meant to include clinically or quantitatively measurable removal of pathogens, including bacteria, germs, viruses, molds, or other susceptible pathogens without killing them. While “decontaminate” can include the removal of 100% of pathogens on the skin or surface, the term refers to any measurable reduction in pathogens on the skin or surface.

“Biocidal,” “biocide,” “anti-microbial,” “sanitize,” and “sanitizer,” are meant to refer to compounds or compositions that measurably kill a pathogen, including bacteria, germs, viruses, molds, or other susceptible pathogens.

Microbiostatic compositions described herein do not kill the microbe, but instead help effectuate its removal from the skin or a hard surface. As used herein “microbiostatic” refers to the rendering of the microbe in a non-infective, but revivable state. The release agent within the composition interferes with the microbe's ability to bind to a surface or skin without affecting either the membrane or the proteins extruded from the microbe thereby aiding in its removal without killing the microbe. Terms such as “bacteriostatic,” “virostatic,” or “fungistatic” are used herein to refer to the same characteristic, i.e., the microbe in a non-infective, but revivable state, as it applied to certain classes of microbes, i.e., bacteria, viruses, and fungus, respectively.

As used herein, the term “release agent” refers to one or more compounds that interfere with the adherence of a microbe to skin or a surface, by either encapsulating the microbe or interrupting its ability to bind to the skin or surface upon which it rests.

As used herein “entangled” broadly describes what happens to the microbe within the soap or decontaminating composition. It does not refer to a specific physical or chemical process, but instead, refers to any interaction between the microbe and the soap or decontaminating composition that disrupts the microbes ability to remain on the skin. “Entangled” can include chemical entanglement, electrostatic entanglement, or physical entanglement of the microbes within the cleansing composition.

“Decontaminating compositions” as described herein are compositions that reduce the pathogen load on a surface upon application of the composition to that surface. Decontamination occurs through the rinsing and removal of the decontaminating composition inclusive of surface microbes that are in stasis. In at least one embodiment, compositions as described herein can be both decontaminating (removal of microbes in stasis) and sanitizing (killing of microbes). Decontaminating compositions can be formulated quite differently from one another and can be used for divergent purposes, for example, cleaning hard surfaces versus soap or cleansing products for application to living tissue, e.g., skin surfaces.

“Excipient” as used herein, refers to the base and other compounds and additives, which together with the release agent(s), make-up the decontaminating composition. According to one embodiment, the excipient is not anti-microbial, i.e., all components are either non-lethal for microbes or are included in an amount that renders them non-lethal for microbes. According to this embodiment, the excipient compositions specifically exclude compounds and materials in in concentrations that are lethal to the pathogen of interest, e.g., virus, bacteria or fungus. As used herein “non-lethal” excipient means that the excipient will not kill a measurable quantity of the microbe which the decontaminating cleanser targets. According to another embodiment, lethal components may be added to the excipient to kill, for example, a desired class or microbes, while maintaining others in stasis. According to this embodiment, the composition may not be as effective as the non-lethal embodiment described above at limiting microbial resistance as any time lethal components are used, the weaker microbes are susceptible to death. However, to the extent the decontaminating composition continues to maintain some portion, percent or class, of the microbes in stasis, improvements in anti-microbial resistance will result.

Unless specifically stated otherwise, excipient refers to the base formulation to which the active ingredients are added and may differ depending upon the type of product that one is making. To the extent the decontaminating composition also includes compounds and materials that are lethal to one or more microbes, these materials will be referred to herein as sanitizing compounds or sanitizing materials and do not form a part of the excipient.

“Protective coating” as used herein refers to the film of material that is left behind on the surface or skin when the decontaminating composition as described herein is removed with water. The protective layer associated with the described decontaminating composition provides hygiene benefits, as well as, skin irritation benefits that will be described in detail below.

The composition, e.g., soap, as described herein comprises one or more release agents that interact with microbes creating an environment where the release agent is simultaneously attracted to the microbe and skin; thereby imparting a positive charge to the skin and the microbe. Since positive charges repel from each other, the microbe is repelled from the skin, allowing the decontaminating composition including the microbes to be rinsed away. As used herein, the term “release agent” refers to one or more compounds that interfere with the adherence of a microbe to skin or a surface, by either encapsulating the microbe or interrupting its ability to bind to the skin or surface upon which it rests. These mechanisms allow the microbe to be repelled from the surface and therefore be washed away; however, since these mechanisms don't interfere with microbe proliferation they are effective, but milder to the skin and to the environment. The release aid causes skin to feel smooth, retain moisture, and reduces the effects of soaps on the skin from repeated washing.

The composition as described herein is effective against most microbes, including but not limited to microbiological organisms, bacteria, viruses, fungi, parasites, and prions. Prions are proteinaceous infectious particles that are responsible for such diseases as transmissible spongiform encephalopathies (TSE) and mad cow disease. Since the compositions do not require specific anti-microbial efficacy with respect to any particular microbe, these compositions can be used effectively against a broad spectrum of microbes or combinations of microbes. For example, a number of microbes are so robust, e.g., C. difficile spores, that they require harsh chemicals to kill them. Such chemicals, while effective, are destructive to the skin and therefore cannot be used in skin contact cleansers. The composition as described herein not only allows removal of the more typical bacteria and viruses, but can also be used to address these robust microbes, since it effectively puts the microbes in stasis allowing them to be removed or rinsed away.

Not wishing to be bound by theory, it is believed that the composition provides a powerful electronic attraction between the microbes and the release agents which is what causes their encapsulation or the interference with their bonding capability. At the same time, the release agent is attracted to the skin surface and forms a substantive coating thereon. The encapsulated microbes and the skin surface both end up positively charged causing the skin to repel the microbes making them easier to remove. The compositions as described use a similar mechanism to the coacervate technology that has been used for decades in conditioning shampoos, simultaneously removing oil and depositing a polymer on the hair. It is believed that the compositions as described herein may be generally characterized as coacervates.

The compositions according to the present disclosure both clean and disinfect hands by removing dirt and germs and microbes. According to one embodiment, the composition as described decreases the transient pathogens on the skin, for example, at least about 50%, for example, at least about 75%, for example at least about 85%, for example, at least about 90%. According to another embodiment, the composition as described can remove for example, 2 times more, for example, 5 times more, for example, 10 times more infective microbes than soap alone. According to yet another embodiment, the composition can remove 80%, for example, 90%, for example 99.9% of germs that cause disease, including difficult to neutralize microbes, such as C. difficile spores.

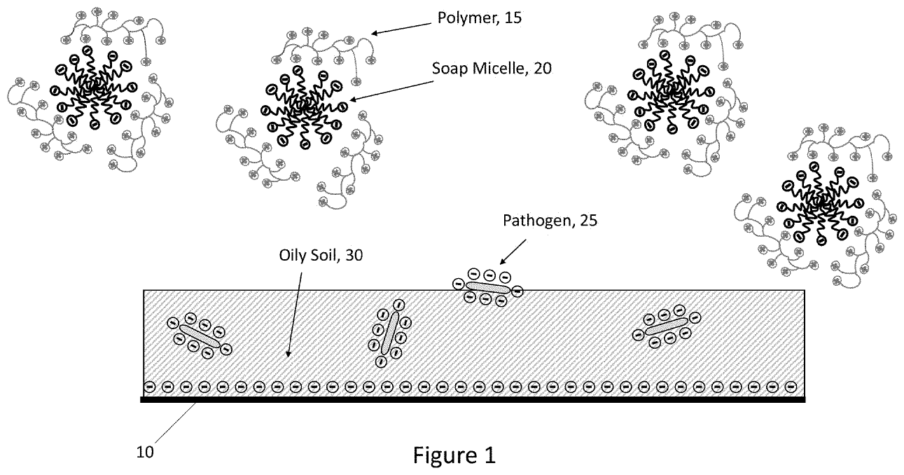

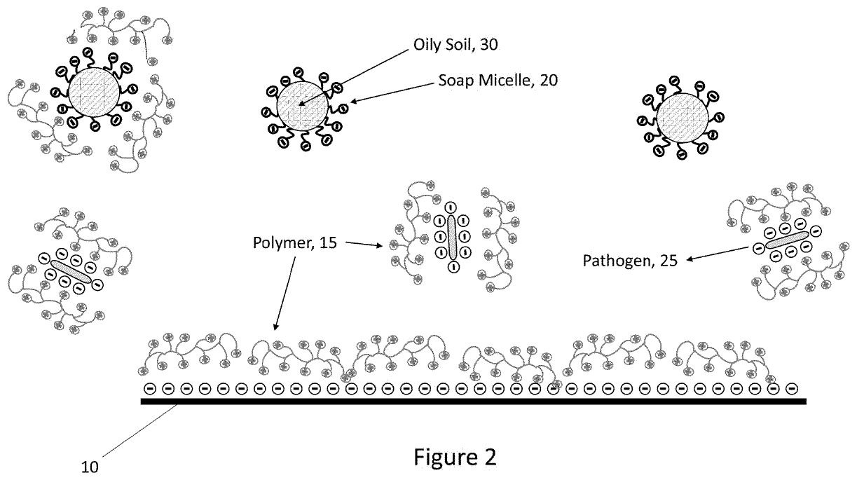

As seen in FIG. 1, a typical skin surface 10 prior to cleaning will have an oily soil layer 30 and microbes 25 attached to the surface. In FIG. 1 the cleansing composition comprising the polymer release agents 15 and the soap 20 as described is theoretically applied to the skin surface 10. According to one embodiment, the polymer 15 and soap micelles 20 would be carried by an excipient base that is, for example, a foam or liquid. The foam or liquid would be applied to the surface 10 of the skin. After application of the cleansing composition, as the surface 10 of the skin is rubbed, the oily layer 30 leaves the skin 10 and the soap 20 and polymer release agents 15 are disrupted as shown in FIG. 2. In the embodiment shown, the oily soil 30 then attaches to the soap micelles 20 while the negatively charged microbes 25 are attracted to the release agents 15. As seen in FIG. 2, the negatively charged pathogens 25 and micelles 20 quickly become surrounded by the positively charged polymeric release agents 15. Additionally seen in FIG. 2, upon release of the oily soil 30 from the surface 10 of the skin, the positively charged release agents 15 are attracted to the negatively charged skin surface 10 and form a substantive layer on the skin 10.

Phosopholipids constitute a substantial portion of both the surface of human skin as well as the cell membranes of most microbes. These phospholipids are tightly packed and carry dense negative charges. Since both the microbes 25 and skin 10 are surrounded by these phospholipid membranes, their negative charges are considerably stronger than the charges on a soap micelle 20. For this reason, the release agent 15 preferentially adsorbs to skin 10 and microbes 25, leaving the soap micelles 20 to be rinsed away.

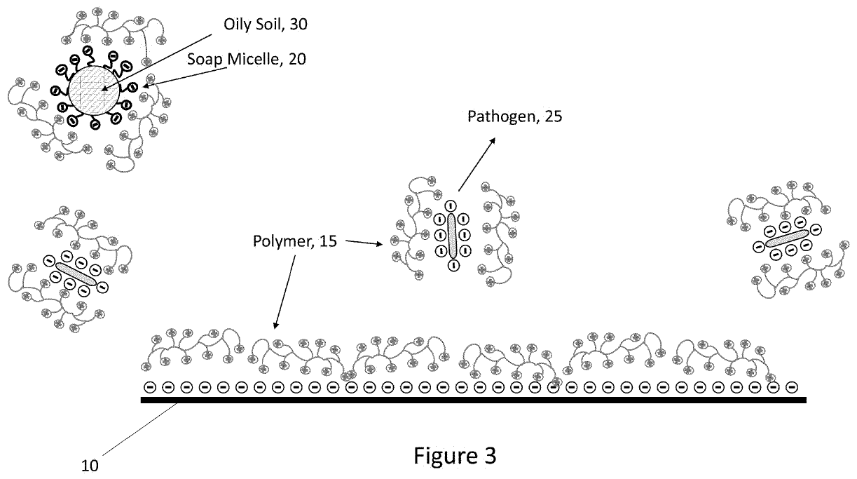

As seen in FIG. 3, once the pathogens 25 and soap micelles 20 including the dirt are entangled with the release agents 15, they become positively charged structures or complexes. These now positively charged structures are ultimately repelled by the positively charged layer of release agent 15 that coats the skin surface 10 making the microbes 25 and dirt easy to simply rinse away. Since the release agents 15 do not disrupt either the membrane or the proteins extruded from the microbe 25, the pathogens 25 are merely removed from the surface 10 and are not killed.

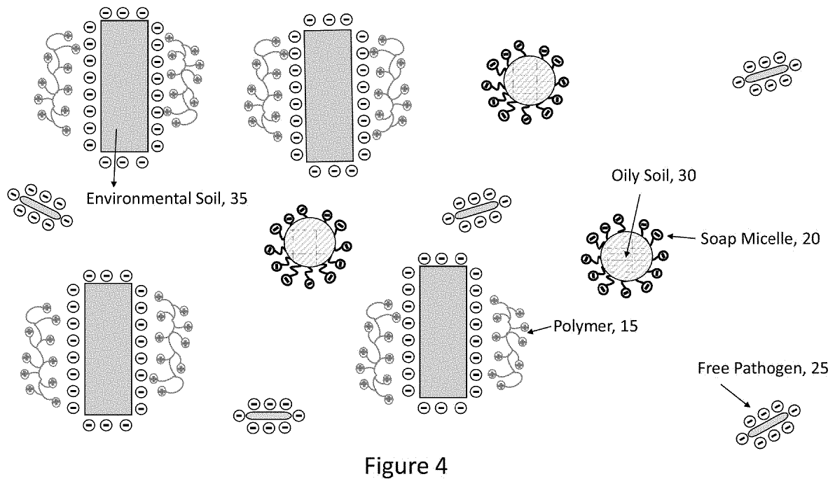

As seen in FIG. 4, after the entangled microbes 25 and release agent 15 are rinsed from the skin surface 10, the release agent 15 and the microbe 25 go down a drain and into a waste water system where there is an abundance of biofilms and soils that will attract the release agents 15 thereby separating the release agent 15 from the microbe 25. The relative concentration of biofilms and soils in the waste system cause the release agent 15 to quickly be disrupted from the microbe 25 much like adsorption or desorption in a chromatography column. This disruption of the microbe/release-agent structures, allows the microbe to come out of stasis and regrow.

The decontaminating compositions as described can be bacteriostatic, fungistatic, a virus inactivator, or a parasite inactivator and are effective against a wide cross-section of microbes. Generally, bacteria and fungus are negatively charged and the described composition is expected to be effective against all classes of both. Viruses and parasites do not necessarily end up in traditional stasis as the bacteria and fungus will; however, the same electrochemical forces work to decontaminated the surface by the removal of the virus or parasite in a manner similar to bacteria and fungus. For purposes of this invention, the entaglement of the virus, parasite or other rmicrobe is considered stasis as defined above.

Viruses, are positively or negatively charged depending on the pH. See, for example, Michen et al., Isoelectric points of viruses, Journal of Applied Microbiology 109(2010) P. 388-397. So, in embodiments of the invention formulated to remove specific viruses, the release agent and pH are selected appropriately to assure entanglement of the virus based upon the expected surface charge of the virus. For viruses having naturally negative surface charges, the decontaminating composition should work effectively, and in the same manner as described herein for both bacteria and fungus.

When removing viruses or parasites, the pH of the formulation should be modified to assure the release agent can effectively capture the microbe. The pH is modified based upon the isoelectric point (IEP). IEP is the pH value at which the net surface charge switches its sign. IEPs for viruses are generally found in the pH range of 2 to 8.5, most frequently between 3.5 and 7. The following table from Michen et al. sets forth the isoelectric points of a number of viruses based upon the reported literature. The table below provides a subsection of the data found in the original table of Michen et al. For information on the tests used to determine the reported values, reference is made to the original article.

| | Isoelectric point |
|---|---|
| Virus | (IEP) |
| Adenoassociated Virus-4 | 2.6 |
| Alstrim Butler | 3.4 |
| Cowpox Brighton | 4.3 |
| Cowpox Brighton (egg) | 4.3 |
| Cowpox Brighton (rabit0 | 4.3 |
| Cowpox Kampen | 5.4 |
| Cowpox Leuwarden | 5.2 |
| Mengovirus L | 8.1 and 4.6 |
| Mengovirus M | 4.4 and 6.3 |
| Mengovirus Mi | 8.4 and 4.6 |
| Mengovirus S | 4.6 and 6.8 |
| Canine parvovirus | 5.0 |
| Hepatitis A virus | 2.8 |
| Human adenovirus 5 | 4.5 |
| Human coxsackievirus B 5 | 4.75 and 6.75 |
| Human echovirus 1 | 5.6 and 5.1 |
| Human echovirus 1 | 4.0 |
| Human echovirus 1 (4CH-1) | 5.5 |
| Human echovirus 1 (R115) | 6.2 |
| Human echovirus 1 (V212) | 6.4 |
| Human echovirus 1 (V239) | 5.3 |
| Human echovirus 1 (V248) | 5.0 |
| Human coxsackievirus A 21 | 6.1 and 4.8 |
| Human rhinovirus 2 | 6.8 |
| Human rhinovirus 2 | 6.4 |
| Influenza A virus H1N1 (Leningrad) | 4.5, 4.35, 4.25, |
| | 4.0 |
| Influenza A virus H3N1 | 6.5-6.8 |
| Influenza A virus H3N2 (Leningrad) | 5.0 |
| Influenza A virus PRB | 5.3 |
| Influenza A virus | 6.5-7.0 |
| Serotype 3 (Dearing) | 3.8 |
| Serotype 3 (Dearing) | 3.9 |
| Monkey pox Chimpanzee Paris | 6.2 |
| Monkey pox Copenhague | 6.5 |
| Monkey pox Denmark | 3.4 |
| Neuro-Vaccinia Levaditi | 4.2 |
| Norwalk virus Funabashi | 5.9 |
| Norwalk virus Hawaii virus | 6.0 |
| Norwalk virus Kashiwa | 5.5 |
| Norwalk virus Narita | 5.5 |
| Norwalk virus | 5.9 |
| Norwalk virus Seto | 6.0 |
| Papillomavirus | 5.0 |
| Poliovirus PV-1 | 7.4 and 4.0 |
| Poliovirus PV-1 | 6.9 |
| Poliovirus PV-1 Brunender | 7.4 and 3.8 |
| Poliovirus PV-1 Brunhilde | 7.1 |
| Poliovirus PV-1 Brunhilde | 7.1 and 4.5 |
| Poliovirus PV-1 Chat | 7.5 and 4.5 |
| Poliovirus PV-1 LSc2ab | 6.6 |
| Poliovirus PV-1 LSc2ab | 6.6 |
| Poliovirus PV-1 LSc2ab | 6.75 and 4.1 |
| Poliovirus PV-1 LSc2ab | 6.75 and 4.5 |
| Poliovirus PV-1 Mahoney | 8.3 |
| Poliovirus PV-2 Sabin T2 | 6.5 |
| Simian Rotavirus A/SA11 | 8.0 |
| Smallpox Butler | 5.7 |
| Smallpox Dijbouti | 5.6 |
| Smallpox Harvey | 5.9 |
| Smallpox Harvey | 3.4 |
| Smallpox Moloya | 5.6 |
| Smallpox Sidi Amock | 5.9 |
| Smallpox Teheran | 5.6 |
| Smallpox Vannes | 5.6 |
| Vaccinia Chaumier | 5.0 |
| Vaccinia Connaught | 4.9 |
| Vaccinia Lister | 5.1 |
| Vaccinia Lister | 3.9 |
| Vaccinia Lister (egg) | 3.7 |
| Vaccinia Lister (rabit) | 3.0 |
| Vaccinia Rabbitpox (Utrecht) | 2.3 |
| Vaccinia WR | 4.8 |
| White cowpox Brighton | 2.8 |
| Whitepocks 64.72.55 | 5.1 |
| Whitepocks 64.72.75 | 4.9 |
| Whitepocks Chimp 9 | 4.8 |
| Whitepocks MK7.73 | 5.3 |
| Whitepocks RZ.10.71 | 5.1 |
| Whitepocks RZ.38.75 | 5.2 |
| Acholeplasma phage O1 | 4.0 |
| Actinomycetes phase MSP8 | 3.5 |
| Bacillus phage ϕ29 | 4.2 |
| Enterobacteria phage GA | 2.1, 2.3 |
| Enterobacteria phage SP | 2.1, 2.6 |
| Enterobacteria phage f2 | 4.0 |
| Enterobacteria phage MS2 | 3.9 |
| Enterobacteria phage MS2 | 3.5 |
| Enterobacteria phage MS2 | 3.1, 3.9 |
| Enterobacteria phage MS2 | 3.9 |
| Enterobacteria phage MS2 | 3.9 |
| Enterobacteria phage MS2 | 2.2, 3.3, |
| | 3.5 |
| Enterobacteria phage PR722 | 3.8-4.2 |
| Enterobacteria phage Qβ | 2.7, 1.9 |
| Enterobacteria phage Qβ | 5.3 |
| Enterobacteria phage T2 | 4.2 |
| Enterobacteria phage T4 | 2.0 |
| Enterobacteria phage T4 | 4.0-5.0 |
| Enterobacteria phage λ C147 | 3.8 |
| Enterobacteria phage μ2 | 4.0 |
| Enterobacteria phage φX174 S13 | 7.0 |
| Enterobacteria phage φX174 Mutants | 7.4 |
| Enterobacteria phage φX174 Wild Type | 6.6 |
| Enterobacteria phage φX174 | 6.0-7.0 |
| Enterobacteria phage φX174 | 2.6 |
| Enterobacteria phage φX174 | 6.6 |
| Enterobacteria phage φX174 | 6.6 |
| PM 2 | 7.3 |
| Pseudomonas phase PP7 | 4.3-4.9 |
| Belladonna mottle virus | 6.3 |
| Cowpea chlorotic mottle virus | 3.8 |
| Erysimum latent virus | 4.7 |
| Red clover necrotic mosaic virus | 5.0 |
| Serotype A | |
| Red clover necrotic mosaic virus | 4.8 |
| Serotype B | |
| Red clover necrotic mosaic virus | 4.6 |
| Serotype C | |
| Scrophularia mottle virus Anagyris | 4.4 |
| Scrophularia mottle virus Czech isolate | 3.9 |
| Scrophularia mottle virus | 4.0 |
| Southern bean mosaic virus variant 1 | 6.0 |
| Southern bean mosaic virus variant 2 | 5.6 |
| Southern bean mosaic virus variant 3 | 5.0 |
| Southern bean mosaic virus variant 4 | 4.0 |
| Tobacco mosaic virus Cucumber virus 4 | 4.9 |
| Tobacco mosaic virus Green aucuba | 4.5 |
| Tobacco mosaic virus Holmes masked | 3.9 |
| Tobacco mosaic virus Holmes rip-gras | 4.5 |
| Tobacco mosaic virus J14D1 | 4.2 |
| Tobacco mosaic virus Ordinary | 3.9 |
| Tobacco mosaic virus Yellow aucuba | 4.6 |
| Turnip yellow mosaic virus | 3.6 |

Viral stasis and reanimation can be evaluated using standard viral assays, for example, plaque assays or focus forming assays.

The compositions as described herein will remove microbes from the skin faster than traditional soaps without the harsh attributes often associated with hand sanitizers or antimicrobial soaps. The improved efficacy means that for people who do not wash thoroughly with soap or other cleaners the compositions as described herein will remove a greater number of microbes than would have otherwise been removed by prior sanitizers or cleansers.

In addition to the encapsulation of microbes and/or interference with their bonding capability, the composition as described provides a protective coating on the skin, while it cleans. This coating property can have multiple effects including protecting the skin from contact with new microbes, reducing the ability of microbes within the pores of the skin to infect others, retaining moisture and/or moisturizers in the skin, and protecting the skin against harsh chemicals that may be found in additional antimicrobial products that are used. The cleansing compositions as described are mild and non-irritating making them particularly useful in skin contact products. While the invention will be discussed in the context of a soap or hard surface cleansers, the concepts are easily translated to other products that would benefit from the same type of microbial entanglement. The cleansing compositions as described can be manufactured in any art recognized form, including liquid, gel, emulsion or foam.

Composition as described comprise one or more release agents that encapsulate or disrupt the attachment between a microbe and the skin or surface. The release agent may be selected from one or more cationic polymeric surfactants or zwitterionic surfactants.

Zwitterionic surfactants are characterized by having two distinct and opposite charges on the molecule at either adjacent or non-adjacent sites. The typical cationic group is a quaternary ammonium group, although other positively charged groups like sulfonium and phosphonium groups can also be used. The typical anionic groups are carboxylates and sulfonates, preferably sulfonates, although other groups like sulfates, phosphates and the like, can be used. Zwitterionic polymers are highly resistant to protein adsorption and bacterial adhesion and exhibit good biocompatibility. Many examples of zwitterionic surfactants are described in the patent literature.

Zwitterionic compounds for use in the described compositions may include betaines, sultaines, phosphobetaines, phosphitaines, including, for example, polybetaine polymers.

Betaine surfactants can include, for example, alkylbetaines such as cocamidopropyl betaines, cocodimethylcarboxymethylbetaine, lauryldimethylcarboxymethylbetaine, lauryldimethylcarboxyethylbetaine, cetyldimethylcarboxymethylbetaine, lauramidopropyle betaine, lauryl-bis-(2-hydroxyethyl) carboxymethylbetaine, oleyldimethylgamma-carboxypropylbetaine, lauryl-bis-(2-hydroxypropyl)-carboxyethylebetaine, and the like.

Sultaines can include, for example, cocamidopropyl hydroxysultaines, cocodimethylpropylsultaine, stearyldimethylpropylsultaine, lauryl-bis-(2-hydroxyethyl) propylsultaine; and am idosultaines, for example, cocoamidodimethylpropylsultaine, stearylamidodimethylpropylsultaine, laurylamidobis-(2-hydroxyethyl) propylsultaine

The phosphobetaines can include lauric-myristicamido-3-hydroxypropylphosphobetaine, cocoamidodidsodium-3-hydroxypropylphosphobetaine, lauric-myristicamidodisodium-3-hydroxypropylphosphobetaine, lauric-myristicam idoglyceryl-phosphobetaine, lauric-myristicamidocarboxydisodium-3-hydroxypropylphosphobetaine, and the like. Phosphitaines can include, for example, cocoamidopropylmonosodiumphosphitaine, lauric-myristicamidopropylmonosodiumphosphitaine and the like.

Cationic polymers for use in the instant composition can include polysaccharides, synthetic cationic polymers, and combinations and copolymers thereof.

Cationic polysaccharides may be composed of one type of sugar or more than one type and may be in straight chain or branched chain geometric arrangements. Cationic polysaccharide polymers can include cationic celluloses and hydroxyethylcelluloses; cationic starches and hydroxyalkyl starches; cationic polymers based on arabinose monomers such as those which could be derived from arabinose vegetable gums; cationic polymers derived from xylose polymers found in materials such as wood, straw, cottonseed hulls, and corn cobs; cationic polymers derived from fucose polymers found as a component of cell walls in seaweed; cationic polymers derived from fructose polymers such as Inulin found in certain plants; cationic polymers based on acid-containing sugars such as galacturonic acid and glucuronic acid; cationic polymers based on amine sugars such as galactosamine and glucosamine; cationic polymers based on 5 and 6 membered ring polyalcohols; cationic polymers based on galactose monomers which occur in plant gums and mucilages; cationic polymers based on mannose monomers such as those found in plants, yeasts, and red algae; cationic polymers based on the galactomannan copolymer known as guar gum obtained from the endosperm of the guar bean.

Synthetic cationic polymers may be produced from ammonium salts, including, but not limited to ammonium chloride, ammonium fluoride, ammonium bromide, ammonium iodine, ammonium bisulfate, ammonium alkyl sulfate, ammonium dihydrogen phosphate, ammonium hydrogen alkyl phosphate, ammonium dialkyl phosphate, and the like. The polymers may be (meth)acrylamides, diallyl dimethyl ammonium salts, acrylic acids, methacrylates, acrylamides, epoxides, Cationic polymers can include diallyl dimethyl ammonium chloride (DADMAC), diallyl dimethyl ammonium fluoride, diallyl dimethyl ammonium bromide, diallyl dimethyl ammonium iodine, diallyl dimethyl ammonium bisulfate, diallyl dimethyl ammonium alkyl sulfate, diallyl dimethyl ammonium dihydrogen phosphate, diallyl dimethyl ammonium hydrogen alkyl phosphate, diallyl dimethyl ammonium dialkyl phosphate, and combinations thereof.

According to one embodiment, cationic quaternary compounds for use in the compositions as described can include any of the poly-quaternary surfactants registered under the International Nomenclature of Cosmetic Ingredients (INCI) as polyquaternium. According to one embodiment, the cationic polymers may be chosen from polyquaterium-2, polyquaterium-7, polyquaterium-44, and polyquaterium-71.

Release agents for use in the described soaps and surface cleaners do not penetrate the cell wall or damage the external proteins. Release agents are those materials that can entangle the microbe causing it to be rendered neutral at the time of the application of the composition to the skin or surface, but which, upon disruption of the microbe/release-agent structure show regrowth of the microbe. This characteristic of the release agent can be easily determined by looking at a sample 24 hours after contact with the release agent and seeing no microbial growth in the solution, however, when wiped across a surface, disrupting the microbe/release-agent entanglement, regrowth occurs. A full description of the methodology for selecting release agents is set forth in the Examples below.

If the decontaminating composition contains a plurality of release agents, the methodology described in the Example would be carried out in the same manner, but on the combination of agents in the expected proportions.

The release agent is included in the composition in an amount of from about 0.1% to about 20%, for example, from about 0.1% to about 5%, for example from about 0.1% to about 3%, for example, from about 0.1% to about 1%, for example from about 0.5% to about 5%, for example from about 1% to about 3%. Unless stated otherwise, all compositional percentages are expressed as weight percents. In traditional soap and surface cleaning products, the concentration of the active ingredients would be modified depending upon the type of cleaner being produced, with non-skin contact cleaners often having higher active levels. In the compositions as described, the concentration of the release agents will remain limited to those concentrations that will not be non-lethal to the microbe of interest. The appropriate range of concentration for each release agent can be determined for bacteria as set forth in the example below. For other microbes, different assaying methods would be used. For example, for viruses, a plaque assay may be used. As with bacteria, other microbes must be in stasis long enough to be removed (minimum concentration), but not killed (maximum concentration).

The release agent may be incorporated into a different excipient depending upon the intended use of the end product. Typical cleaning solutions are water based, but the release agent as described may be used in other systems, including for example, oil-based, or propellant-based systems or emulsions.

The characteristics of the excipient may vary widely. Formulations for surface cleaners and skin cleaners are well reported in the art. Any art recognized formulation would be used as an excipient in conjunction with the described invention to the extent that the excipient does not include additives or additive levels that are lethal to microbes. For example, the pH of surface cleaners and soaps are known to differ, the use of additional sanitizing actives may differ between a surface cleaner and soap, the use of moisturizers, lubricants and other emollients with differ, as can the amount of release agent(s).

As defined herein, the non-active excipient can include, dyes, moisturizing agents, skin conditioning agents, thickeners, solvents, vitamins, anti-oxidants, pH modifiers, film formers, anti-inflammatories, abrasives, colorants, humectants, emollients, fragrances, and botanical extracts. Again, anything added to the excipient would have to be non-lethal to the microbes. For example, it is known that certain botanicals have anti-microbial properties, so the addition of those botanical would have to be limited to low levels.

The decontaminating compositions as described herein may optionally comprise one or more surfactants chosen from amphoteric surfactants, anionic surfactants, cationic surfactants or nonionic surfactants which are often included as cleansers. These surfactants may be used in the excipient to the extent they do not interfere with the release agents and are non-lethal to the microbes. According to one embodiment, the decontaminating composition maintains a net positive charge.

Optional amphoteric surfactants for use in the decontaminating composition as described include, but are not limited to, dodecyl/dimethyl amine oxide marketed under the tradename AMMONYX LO from Stepan Co. and cocamidopropyl betaine marketed under the tradename AMPHOSOL HCP-HP both from Stepan Co. Appropriate amphoteric surfactants are readily available and are marketed by companies such as Akzo Nobel, Pilot and Solvay Chemical. Amphoteric surfactant may be present in the decontaminating composition in an amount of from about 0.01% to about 20%, for example, from about 0.05% to about 10%, for example, from about 0.1% to about 5%.

Other surfactants including anionic, nonionic and cationic may optionally be included. Anionic surfactants are defined as those surfactants that possess a negative charge and include such surfactant classes as sulfates, including for example, sodium laureth sulfates, ammonium lauryl sulfates, and monoglyceride sulfates, sulfonates, including for example alkyl benzene sulfanates, and fatty glycerol and ether sulfanates, sulfosuccinates, taurates, including for example acyl methyl taurates, isethionates, alkanoic acids, ester carboxylic acids and ether carboxylic acids.

Nonionic surfactants are defined as those surfactants possessing no charge moieties within the molecular structure and include such surfactant classes as alkanol amines, alkanolamides, ethoxylated amides, ethoxylated fatty acids, ethoxylated fatty alcohols, alkoxylated esters, alkyl polyglucosides, alkoxylated triglycerides, sorbitan esters and sorbitan ethers.

Cationic surfactants are defined as those surfactants that possess a positive charge and include such surfactant classes as benzalkonium, stearalkonium, and centrimonium chlorides, trimethyl ammoniums, and methyl sulfates.

The anionic, cationic and non-ionic surfactants can be present in the decontaminating composition in an amount of from about 0% to about 20%, for example, from about 0.01% to about 15%, for example, from about 0.01% to about 12%, for example, from about 2% to about 12%, for example from about 3% to about 10%, for example from about 4% to about 7%, for example, from about 0.1% to about 6%.

Optional ingredients that may also be added to the formulation include, for example, emollients, fragrances, dyes, humectants, moisturizing agents, skin conditioning agents, chelating agents, preservatives, thickeners, solvents, botanicals, vitamins, anti-oxidants, pH modifiers, film formers, anti-inflammatories, abrasives, colorants, and the like.

Depending upon the embodiment, optional stabilizers may be used to inhibit reactions between ingredients and to maintain the homogeneity of the composition. According to one embodiment, if the decontaminating composition is a foaming composition, it may include one or more foam stabilizers. Suitable foam stabilizers can be chosen from foam boosters, alkyl polyglucosides, amphoteric surfactants, nonionic surfactants, amide oxides. The stabilizer will be present in the decontaminating composition in an amount of from about 0% to about 10%, for example from about 0.01% to about 5%, for example, from about 0.01% to about 2%.

Appropriate solubilizers for use in the decontaminating compositions as described will be readily apparent to the skilled artisan and can include hydrotropes, nonionic, surfactants, chelating agents, builders. The solubilizer can be present in the decontaminating composition in an amount of from about 0% to about 5%, for example, from about 0% to about 2.0%, for example, from about 0.1% to about 2.0%.

Generally, emollients lubricate, soothe, and soften the skin surface. Exemplary emollients include silicons, ethoxylated or propoxylated oily or waxy ingredients such as esters, ethers, fatty alcohols, hydrocarbons, lanolin, and the like. The emollients can be present in the decontaminating composition in an amount of from about 0% to about 10%, for example, from about 0.1% to about 3%, for example, from about 0.05% to about 1%.

Humectants are hydroscopic agents that are widely used as moisturizers. Their function is to prevent the loss of moisture from the skin and to attract moisture from the environment. Common humectants include, for example, sodium PCA, glycerin, propylene glycol, butylene glycol, betaine, sodium hyaluronate, sorbitol, urea, hydroxyethyl urea, and the like. The humectants can be present in the decontaminating composition in an amount of from about 0% to about 5.0%, for example, from about 0.1% to about 2.5%, for example, from about 0.5% to about 1.5%.

Preservatives for increasing the shelf life of the decontaminating composition may also be used. Exemplary suitable preservatives include, but are not limited to disodium EDTA; tetrasodium EDTA; iodopropynyl butylcarbamate; benzoic esters (parabens), such as methylparaben, propylparaben, butylparaben, ethylparaben, sodium methylparaben, and sodium propylparaben; phenoxyethanol; benzyl alcohol; phenethyl alcohol; amidazolidinyl urea; diazolidinyl urea; citric acid, lactic acid, kathon, phenoxyethanol, 2-bromo-2 nitro-propane-1,3,-diol, potassium sorbate, and the like. The preservatives can be present in the decontaminating composition in an amount of from about 0.01 to about 3%, for example, from about 0.01% to about 1%, for example, from about 0.01% to about 0.08%, for example from about 0.02% to about 0.08%, for example from about 0.02% to about 0.1%.

According to one embodiment, the preservative is chosen from EDTA. The inclusion of EDTA has a secondary effect in this decontaminating composition. EDTA is a chelating agent and can bind iron. When the iron is bound so too is oxygen which starves the microbe temporarily reducing the activity of the microbe during washing. Other chelating agents may be included to achieve the same effect.

Suitable skin conditioning agents include, for example, hydrolyzed plant proteins such as hydrolyzed wheat protein, hydrolyzed soy protein, hydrolyzed collagen, and the like. The skin conditioning agents can be present in the decontaminating composition in an amount of from about 0% to about 10%, for example, from about 0.1% to about 5%, for example, from about 0.5% to about 3%.

The pH of the system is maintained from about 3 to about 13. Art recognized acids and bases may be used to modify the pH for the desired end product. For skin applications, the pH will generally be neutral, from about 4 to about 9, for example, about 4 to about 7, for example about 4 to about 6, for example, from about 4.5 to about 5.5; however, when formulating hard surface cleaners or other cleansers that will not contact the human body, the pH may be raised or lowered as appropriate. Typical hard surface cleaners will have a pH in the range of about 9.5 to about 13, for example, about 9.5 to about 11, for example, about 9.5 to about 10.5. pH modifiers include both basic and acidic pH modifiers. Some examples of basic pH modifiers that may be used in the decontaminating compositions of the present disclosure include, but are not limited to, ammonia; sodium, potassium, and lithium hydroxide; sodium, potassium, and lithium metal silicates; monoethanolamine; triethylamine; isopropanolamine; ethanolamine; and triethanolamine. Acidic pH modifiers that may be used in the formulations of the present disclosure include, but are not limited to, mineral acids; carboxylic acids; and polymeric acids, including by way of example, citric acid. The pH modifiers will be used in an amount necessary to achieve the desired pH. For example, the pH modifiers can be present in the decontaminating composition in an amount of from about 0% to about 5%, for example, from about 0.01% to about 3%, for example, from about 0.01% to about 2%, for example from about 0.010% to about 1%, for example, from about 0.01% to about 0.5%, for example, from about 0.01% to about 0.05%. According to one embodiment, the decontaminating composition has a pH in the alkaline range. According to another embodiment, the decontaminating composition has a pH in the neutral/acidic range.

A chelating agent is a substance whose molecules can form one or more bonds with a metal ion. In particular, water that may be contained in the decontaminating composition often contains metal ions, such as calcium ions, that might react with anionic components (e.g., acids) present within the composition. Some examples of chelating agents that may be used in the decontaminating composition of the present disclosure include, but are not limited to, ethylenediamines, ethylenediaminetetraacetic acids (EDTA) acids and/or salts thereof, for example, tetrasodium EDTA, citrate, pyrithione, N,N′-bis(o-hydroxybenzyl)ethylenediamine-N,N′diacetic acid; ethylenebis-N,N′-(2-o-hydroxyphenyl)glycine, 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid); N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; nitrilotris(methylenephosphonic acid); and triethylenetetramine-N,N,N′,N″,N′″,N′″-hexaacetic acid, glucuronic acids and/or salts thereof, succinic acid and/or salts thereof, polyphosphates, organophosphates, and the like. The chelating agent can be present in the decontaminating composition in an amount of from about 0% to about 5%, for example, from about 0.01% to about 3%, for example, from about 0.05% to about 2%.

Fragrances and dyes may be used in the decontaminating compositions as appropriate to appeal to the purchasing consumer. Fragrances and dyes can be present in the decontaminating composition in an amount of from about 0% to about 5%, for example, from about 0.1% to about 1%, for example, from about 0.1% to about 0.5%.

Moisturizing agents for use in the decontaminating compositions as described can include, but are not limited to collagen; lecithins; liposomes; peptides; polysaccharides; glycerin; sorbitol; propylene glycol; calcium pantothenate; urea; caprylyl glycol; butylene glycol; glucose; magnesium lactate; potassium chloride; potassium lactate; ethylhexylglycerin; dipropylene glycol; silicones, such as dimethicone and cyclomethicone; fatty acids, for example, lanolin acid; fatty alcohols, for example, lanolin alcohol; hydrocarbon oils and waxes; petrolatum; polyhydric alcohols; sterols, for example, cholesterol; vegetable and animal fats, for example, cocoa butter, vegetable waxes, carnauba wax, wax esters, and bees wax; hyaluronic acid, ceramics; caprylic/capric triglycerides; magnesium aspartame; potassium aspartame; sarcosine; and the like. The moisturizing agent can be present in the decontaminating composition in an amount of from about 0% to about 10%, for example, from about 0.1% to about 5%, for example, from about 0.5% to about 3%.

Thickeners for use in the decontaminating composition as described include, for example, cetyl alcohol, stearyl alcohol, carnauba wax, and stearic acid, carboxyethyl cellulose, carboxymethyl cellulose, guar gum, xanthan gum, gelatin, silica, bentonite, silicates, carbomer polymers, and the like. Thickeners can be present in the decontaminating composition in an amount of from about 0% to about 5%, for example, from about 0.1% to about 3%, for example, from about 0.2% to about 1%.

Botanicals for use in the decontaminating compositions as described may include, for example, aloe vera, green tea extract, cucumber extract, chamomile, oat, Aspen Bark, Bamboo Leaf, Banaba Leaf, Burdock Root, Chamomile, Chrysanthemum, Cucumber Peel, Ginkgo Biloba Leaf, Ginseng Root, Grape Seed, Green Tea, Honey Suckle Flower, Horse Chest Nut, Licorice Root, Maca, Milk Thistle (Silymarin), Olive Leaf, Rosehips, Rosemary, Sacha Inchi, Sea Buckthorn, Sunflower, Thyme, White Willow Bark, and the like. Botanicals can be present in the decontaminating composition in an amount of from about 0% to about 5%, for example, from about 0.1% to about 3%, for example, from about 0.1% to about 1%.

Vitamins for use in the decontaminating composition may include for example, Vitamins A, B, C, D, E, tocopheryl acetate, retinyl palmitate, panthenol, and ascorbic acid. Vitamins can be present in the decontaminating composition in an amount of from about 0% to about 5%, for example, from about 0.1% to about 3%, for example, from about 0.001% to about 1%.

Antioxidants for use in the decontaminating composition as described can include one or more of Glutathione, superoxide dismutase, ubiquinone, omega-fatty acids, Vitamin C, Beta-Glucan, Thioctic Acid, Magnesium Ascorbyl, Phosphate, Ferulic Acid, Superoxide Dismutase, Epigallocatechin Gallate, Ergothioneine, Glutathione, Xanthophylls, and the like. Antioxidants may be present in the composition in an amount of from about 0% to about 5%, for example, from about 0.001% to about 3%, for example, from about 0.001% to about 1%.

The artisan skilled in the formulation of soaps or hard surface cleaners understands that ingredients may be selected to provide more than one function in a composition. Thus, a single ingredient may be chosen to act, for example, as a pH modifier and a preservative, or as a moisturizer and as a humectant.

According to a second embodiment, and under appropriate conditions sanitizing agents may be added to the composition as described. For example, if the surface to be cleaned includes both bacteria and viruses. If one wanted to kill one class of pathogens but not the other, then the release agents as described can be used in concert with an antimicrobial that is lethal to only one of the pathogens present. Other compatible active agents include, but are not limited to non-ionic surfactants, hydrotropes, chelating agents, preservatives, alcohols, e.g., ethanol, and biocidally active botanical extracts, for example, essential oils, and like. These compatible active agents may be present in an amount of from about 0.01% to about 10%, for example, from about 0.05% to about 5%, for example, from about 0.1% to about 1%. According to this embodiment, the decontaminating composition may not be as effective as the primary embodiment described at limiting microbial resistance as any time a sanitizing composition is used, the weaker microbes are susceptible to death.

The compositions as described herein can be used in any type of cleanser, including but not limited to, soaps, shampoos, body wash, personal care items, including, lip balms, lipstick, skin protectants, feminine hygiene products, personal lubricants, disinfecting wipes, baby wipes, hard surface cleaners, air fresheners, and the like. The compositions as described will be particularly effective in health care environments, elderly care, day care, schools, or any environment where people regularly congregate and come into contact with germs.

The cleansing composition creates the same substantive coating on hard surfaces that are cleaned with it. The coating on a hard surface may seal microbes within the pores of the surface rendering them less effectively transferred between the hard surface and something or someone that comes into contact with the surface. Hard surface cleaning compositions may optionally contain perfumes, fragrances, fragrance release agents, dyes, colorants, stabilizers, thickeners, defoamers, lubricants, odor control agents, bleaching agents, acids, bases, preservatives, hydrotropes, chelating agents, surfactants or other polymers.

Hard surface cleaners may be applied by any art recognized method including from a dispenser, a spray device, an aerosolizer, a roller, a pad, a wipe or a wiping implement.

### EXAMPLES

### Example 1—Identifying Appropriate Release Agents and Concentrations

Release agents for use in the described soaps and hard surface cleaners are those materials that can coat all or part of a microbe or spore wall, but not penetrate the cellular or spore wall, rendering the microbe unable to infect skin or reproduce at the time of application. Not only does the release agent coat the microbe, it also coats skin. Because the microbe coating and skin coating have similar charges, the microbe is repelled from the skin surface into the soap layer. After rinsing the soap, the material is disrupted and releases, allowing regrowth of the microbe. The material has little or no toxicity to the microbe during the application period and does not impact the microbe population so it reduces pathogen counts like antimicrobial soaps without increasing antibiotic resistance which may be caused by antimicrobial soaps. The characteristic of the polymer at the lower limit of activity is easily demonstrated by looking at a sample 24 hours after contact with the release agent and seeing no microbial growth in the solution. However, when the solution is swabbed onto a growth media plate, polymer disruption and release occur, allowing the microbe to resume growth. At the upper bacteriostatic material limit, after 2 minutes—a time slightly longer than hand washing—the solution shows no initial growth in solution but growth after swabbing onto a growth media plate.

A series of compounds were tested to determine whether microbes exposed to these compositions and rinsed off were still viable to regrow.

The standard MIC protocol (Internal NTC Microbiology Lab Method: MB-025) was followed for this work with some modifications. The organisms used were Staphylococcus aureus ATCC® 6538™ and Escherichia coli ATCC® 15225™ (94 TFM). All organisms were purchased from Microbiologics® and grown in the lab on Trypticase Soy Agar (TSA) at 35° C. The organisms were washed off of the agar into 0.1% peptone water and visually matched to a McFarland 0.5 Standard (equal to 108 cfu/mL).

Ten-fold serial dilutions were prepared for all chemicals tested in Mueller-Hinton II Broth (MH Broth). An initial 1:10 dilution in MH Broth was made and all subsequent dilutions were prepared by removing 1 mL from the previous dilution and transferring to a sterile MH Broth tube. For the initial screening, 1:10 serial dilutions were prepared through 1:1,000,000. One mL of the bacterial solution was added to each dilution tube and vortexed to mix. All samples were then incubated for 18±4 hours at 37° C. and then all tubes were observed for growth (turbidity) in the tube.

For subsequent round(s) of testing, the parts per million (ppm) of chemical was calculated and dilution tubes were prepared to bracket the ppm of growth/no-growth seen during the first round of testing. Each tube was prepared by adding in the calculated amount of MH Broth and test substance. The inoculation and incubation procedure was followed as stated above.

After incubation, all tubes were observed for growth. For the second round of samples, each tube with no growth was streaked onto a Trypticase Soy Agar (TSA) plate to identify if the organism was still present in the tube but unable to grow in the presence of the chemical. All TSA plates were incubated for 18±4 hours at 37° C. and then observed for growth/no-growth.

The bateriostatic MIC was identified where no growth was observed in the tube and growth was observed on the plate.

TABLE 1

| | | Upper | | |
|---|---|---|---|---|
| | | Limit | Lower | |
| | | from | Limit from | |
| | Actives | 1 min | 24-hour | |
| Release Agent | W % | Exposure | growth (%) | Comment |
| Rewocare 755 | 40.0 | 36.0 | 4.0 | |
| Modified Cornstarch | 22.0 | 20.0 | 1.0 | |
| 1-Propanaminium, | 20.0 | 18.0 | 2.0 | |
| N,N,N-trimethyl-3- | | | | |
| [2-methyl-1-oxo- | | | | |
| 2-propen-1-yl)amino]-, | | | | |
| chloride (1:1), | | | | |
| polymer with ethyl-2- | | | | |
| propenoate | | | | |
| and sodium 2- | | | | |
| propenoate (1:1) | | | | |
| Polyquaternium-71 | 68.0 | 7.0 | 3.4 | |
| Polyquaternium-44 | 12.5 | 12.0 | 1.5 | |
| Polyquaternium-7 | 9.0 | 8.0 | 0.4 | |
| Polyquaternium-2 | 62.0 | 56.0 | 0.6 | |
| Disodium | 40.0 | 36.0 | 2.0 | |
| Cocamphodiacetate | | | | |
| Cocamidopropyl | 30.0 | 27.0 | 1.0 | |
| betaine | | | | |
| Cocamidopropyl | 50.0 | 45.0 | 0.5 | |
| hydroxy sultaine | | | | |
| Lauramidopropyle | 36.5 | 4.0 | 0.1 | |
| betaine | | | | |
| Sodium-Bis- | 40.0 | 4.0 | 4.0 | Range too |
| Hydroxyethylglucinate | | | | narrow, but |
| Coco Glucosides | | | | may be |
| Crosspolymer | | | | useful in |
| | | | | mixed |
| | | | | system |
| Dioctyl sulfosuccinate | 49.0 | 5.0 | 5.0 | Range too |
| sodium salt | | | | narrow, but |
| | | | | may be |
| | | | | useful in |
| | | | | mixed |
| | | | | system |
| Sokalan HP-20 | 80.0 | 72.0 | 32.0 | Too high |
| | | | | for safety |
| Vinyl pyridine with | 40.0 | 36.0 | 12.0 | Too high |
| betaine | | | | for safety |

### Example 2

A composition for cleaning skin can be prepared by combining the following ingredients in either liquid or foam form.

| Ingredient | Function | Level (wt. %) |
|---|---|---|
| Water | Solvent | QS |
| Sodium Laureth Sulfate | Cleanser | 6.00 |
| Cocamidopropyl Hydroxysultaine | Cleanser | 6.00 |
| Citric Acid | pH adjuster | 0.03 |
| Kathon CG | preservative | 0.08 |
| Polyquaternium-2 | Coats skin | 5.00 |
| | and | |
| | pathogens | |

Users will dispense approximately 15 mL of foam (20:1 air to handwash) or about 1.8 mls liquid hand wash into cupped hands and wash in a vigorous manner for approximately 30-60 seconds, followed by rinsing the hands with water and drying. As described below, the described product has two modes of action, neither of which achieves its primary intended purposes through chemical action within or on the body. The product's first mode of action is to physically remove pathogens from the skin during handwashing. It achieves this action by means of its positively charged polyquarternium-2 polymer ingredient, which loosely attaches to the negatively charged pathogens and skin. The polymer coats the skin (changing its charge to positive) and physically wraps itself around the pathogen so the pathogen can no longer attach to the positively charged skin. As the handwash is rinsed with water, it along with the attached soil and pathogens are rinsed down the drain where the pathogens will be released unchanged from the polymer when diluted with water. Some polymer will remain on the hands after rinsing, which provides a film cosmetically protecting the hands from repeated washing, analogous to the coating of hair in hair conditioner products. Eventually, as the oily layer reforms on the skin, the polymer is released from the skin surface.

The product's other mode of action is to, along with water, physically clean the hands. It achieves this mode of action by the use of its sodium laureth sulfate and cocamidopropyl hydroxysultaine ingredients. Sodium laureth sulfate is an anionic detergent and surfactant and cocamidopropyl hydroxysultaine is an amphoteric surfactant that boosts the foaming capability of sodium laureth sulfate. These ingredients are used in the product to emulsify the oily soil on the hands and to facilitate the mechanical removal of debris from the skin.

Other embodiments of the present invention can include alternative variations. These and other variations and modifications will become apparent to those skilled in the art once the above disclosure is fully appreciated. It is intended that the following claims be interpreted to embrace all such variations and modifications.

1. A microbiostatic cleansing composition comprising:
an excipient; and
at least one release agent that entangles one or more microbes upon contact placing the microbe in stasis.

2. The microbiostatic cleansing composition of claim 1, wherein the release agent is present in the cleansing composition at a concentration such that
when the composition is contacted with a microbe in solution, the combined solution shows no initial growth of the microbe after two minutes, but the combined solution shows growth of the microbe after swabbing solution onto a growth media plate.

3. The microbiostatic composition of claim 1, wherein the release agent is chosen from one or more cationic polymeric surfactants or zwitterionic surfactants.

4. The microbiostatic composition of claim 1, wherein the release agent is chosen from one or more zwitterionic polymers.

5. The microbiostatic composition of claim 1, wherein the release agent is chosen from one or more cationic polymers.

6. The microbiostatic composition of claim 1, wherein the excipient comprises one or more of dyes, moisturizing agents, skin conditioning agents, thickeners, solvents, vitamins, anti-oxidants, pH modifiers, film formers, anti-inflammatories, abrasives, colorants, humectants, emollients, fragrances, and botanical extracts.

7. The microbiostatic composition of claim 6, wherein the cleansing composition is a hard surface cleaner having a pH of from about 9.5 to about 13.

8. The microbiostatic composition of claim 1, wherein the cleansing composition is a skin cleaner having a pH of from about 4.5 to about 5.5.

9. The microbiostatic composition of claim 1, wherein the composition is bacteriostatic.

10. The microbiostatic composition of claim 1, wherein the composition is fungistatic.

11. A microbiostatic cleansing composition consisting essentially of:
a release agent chosen from one of a zwitterionic surfactant and a cationic polymeric surfactant that entangles one or more microbes upon contact, but which upon disruption of the entanglement releases the microbe(s) without killing it; and
an excipient.

12. A bacteriostatic cleansing composition consisting essentially of:
an excipient; and
at least one release agent that entangles one or more bacteria upon contact, but which upon disruption of the entanglement releases the bacteria to be revived.

13. The bacteriostatic cleansing composition of claim 12, wherein the bacteria are chosen from one or more of Staphylococcus aureus, Streptococcus pyogenes, Clostridium difficile, and Clostridium botulinum, Salmonella, Escherichia coli, Klebsiella, Haemophilus, Pseudomonas aeruginosa, Proteus and Shigella dysenteriae.

14. A bacteriostatic skin cleansing composition for the removal of C. difficle comprising:
an excipient having a pH of from about 4.5 to about 7; and
at least one release agent chosen from one or more cationic polymeric surfactants or zwitterionic surfactants wherein the release agent is present in a concentration to place the C. difficle in stasis.

15. The bacteriostatic skin cleansing composition of claim 14, wherein the release agent is chosen from one or more cationic polymer surfactants.

16. The bacteriostatic skin cleansing composition of claim 14, wherein the release agent is present in the cleansing composition at a concentration such that
when the composition is contacted with C. difficle in solution, the combined solution shows no initial growth of C. difficle after two minutes, but the combined solution shows growth of C. difficle after swabbing solution onto a growth media plate.

17. A method of cleansing skin comprising:
applying a cleansing composition comprising a excipient and at least one microbial release agent that entangles microbes upon contact, but which upon disruption of the entanglement releases the microbe(s) without killing them; and
rinsing the cleansing composition along with the microbes in stasis off of the skin surface with water.

18. The method of claim 17, further comprises depositing a protective layer on the skin.

19. The composition of claim 1, wherein the excipient is non-lethal.
